(19) United States
(12) Patent Application Publication
(10) Pub. No.: US 2025/0375645 A1
(43) Pub. Date: Dec. 11, 2025

(54) APPLICATION OF LOIGOLACTOBACILLUS CORYNIFORMIS SUBSP.CORYNIFORMIS IN EFFICIENT DEGRADATION OF AFLATOXIN

(71) Applicant: Zhejiang University of Technology, Hangzhou (CN)

(72) Inventors: Yan Wang, Hangzhou (CN); Yufei Wang, Hangzhou (CN); Qing Zhang, Hangzhou (CN); Hao Yan, Hangzhou (CN); Jing Neng, Hangzhou (CN); Kai Yang, Hangzhou (CN); Ming Cai, Hangzhou (CN)

(73) Assignee: Zhejiang University of Technology, Hangzhou (CN)

(21) Appl. No.: 19/310,863

(22) Filed: Aug. 26, 2025

(30) Foreign Application Priority Data
Aug. 29, 2024 (CN) 202411196229.7

(51) Int. Cl. A62D 3/02; A62D 101/28; C12N 1/20; C12R 1/225

(52) U.S. Cl. CPC A62D 3/02; C12N 1/20; A62D 2101/28; C12R 2001/225

(57) ABSTRACT

The present disclosure discloses an application of Loigolactobacillus coryniformis subsp. coryniformis in the efficient degradation of aflatoxin. The method is as follows: Loigolactobacillus coryniformis subsp. coryniformis 523L5 is inoculated into LB broth containing Aflatoxin B1, and Aflatoxin B1 is degraded by dark reaction at 25-55° C. and pH 3.0-9.0. The Loigolactobacillus coryniformis subsp. coryniformis 523L5 has good growth performance, high acid production ability, strong salt tolerance and acid tolerance, and may tolerate about 7% salinity. It may grow under the condition of pH 4.5 and has good fermentation performance. The fermentation broth can efficiently degrade Aflatoxin B1 under acidic (pH 5.0-7.0) and high temperature (50-55° C.) conditions, and the highest degradation rate can reach 88.13% at 40° C. and pH 7.0.

### TECHNICAL FIELD

The present disclosure belongs to the field of microbial detoxification, specifically, an application of Loigolactobacillus coryniformis subsp. coryniformis in an efficient degradation of aflatoxin.

### BACKGROUND

Aflatoxins are secondary metabolites with biological activity, produced by Aspergillus species such as Aspergillus flavus and Aspergillus parasiticus, which are polyketides. As one of the five major mycotoxins, aflatoxin is the main source of grain and food contamination. It is widely found in food crops such as wheat, corn, sorghum, peanuts, soybeans, cassava, spices, fruits, milk, meat, fermented products, and feeds. Among them, Aflatoxin B1 (AFB1) is the most widely contaminated and most toxic toxin, with nephrotoxicity, hepatotoxicity, and immunotoxicity. Meanwhile, it also has carcinogenicity and teratogenicity, and is classified as a group I carcinogen by the International Agency for Research on Cancer (IARC) of the World Health Organization (WHO). The extensive contamination and toxicity of AFB1 seriously threaten the health and safety of humans and animals and the development of agriculture. Therefore, there is an urgent need for strategies for AFB1 removal.

Traditional physical methods such as high temperature, non-thermal methods such as non-thermal plasma, electron beam radiation and pulsed light, and physical adsorption are safe and reliable but have poor degradation effects on toxins (Yousefi M, Mohammadi M A, Khajavi M Z et al., Application of novel non-thermal physical technologies to degrade toxins, Journal of fungi (Basel), 2021.). Chemical methods are used to convert toxins through chemical reactions. Common methods include hydrolysis, ammonification, and redox (Pankaj S K, Shi H, Keener K M, A review of novel physical and chemical decontamination technologies for aflatoxin in food. Trends in Food Science & Technology, 2018.). However, treated materials will retain chemical substances, and treatments affect the nutritional value of food, and even produce secondary toxic effects. Biodegradation refers to the interaction of microorganisms and certain substances produced by them with toxins, changing their original structure and transforming them into low-toxic or non-toxic substances (Guan Y, Chen J, Nepovimova E et al., Aflatoxin Detoxification Using Microorganisms and Enzymes. Toxins, 2021.). In recent years, biological detoxification technology has gradually become popular. Compared with physical and chemical detoxification methods, biological detoxification is safer and effective, and can retain the nutritional value and flavor quality of food.

Lactic Acid Bacteria (LAB) are a class of microorganisms that widely exist in nature. They have important applications in the food industry, especially in the production of fermented foods. In recent years, some studies have found that some lactic acid bacteria (including Lactobacillus plantarum, Lactobacillus rhamnosus, Lactobacillus kluyveri, etc.) can remove aflatoxin. The main mechanisms include cell wall adsorption and degradation of metabolites. The cell wall adsorption refers to the interaction between peptidoglycan, carbohydrate, phosphate, and other components on the cell wall and the functional groups of Aflatoxin B1. It binds to toxins through physical adsorption, ion exchange, and complexation (Asurmendi P, Gerbaldo G, Pascual L, Barberis L., Lactic acid bacteria with promising AFB 1 binding properties as an alternative strategy tomitigate contamination on brewers' grains. Journal of Environmental Science and Health Part B-Pesticides Food Contamin, 2020.). Organic acids are metabolites produced by lactic acid bacteria. Antifungal activity is achieved by inhibiting fungal metabolic activity (Saelim K, Jampaphaeng K, Maneerat S., Functional properties of Lactobacillus plantarum S0/7 isolated fermented stinky bean (Sa Taw Dong) and its use as a starter culture. Journal of Functional Foods, 2017.). In addition to organic acids, lactic acid bacteria also produce antimicrobial peptides, which destroy the integrity of the cell membrane by interacting with lipids on the cell membrane. (Muhialdin B J, Algboory H L, Kadum H, et al., Antifungal activity determination for the peptides generated by Lactobacillus plantarum TE10 against Aspergillus flavus in maize seeds. Food Control, 2020.). In addition, the fermentation supernatant of some lactic acid bacteria significantly reduces the detoxification activity after protease K treatment, indicating that the fermentation supernatant contained certain proteins or enzymes, which may degrade Aflatoxin B1 into non-toxic substances (Yingchao Z, Peng W, Qing K, Peter J, Biotransformation of Aflatoxin B1 by Lactobacillus helviticus FAM22155 in wheat bran by solid-state fermentation. Food Chemistry, 2021.)

A patent named “Strain of degrading Aflatoxin B1 strain S262 and its application” (CN113430128A) by Liu Na et al. provides a strain of Bacillus sonora S262 isolated from pig manure. The degradation rate of Aflatoxin B1 is 45.5% after 24 h, and the degradation rate is 84.32% after 72 h. It is necessary to increase the temperature or prolong the degradation time to have a better degradation effect. A strain of Bacillus amyloliquefaciens ZJSY6 isolated from soil is screened from the patent “A strain for degrading aflatoxin and its culture and application” (CN117004504A) by Zhang Xiaojing et al. The degradation rate of Aflatoxin B1 is 55.87% after 24 h, and the degradation rate is 87.72% after 72 h. The strain shows positive results in positive experiments with a pH of 5.7, but it is necessary to extend the degradation time to have a better degradation effect. The strains mentioned in the above patents may not efficiently degrade Aflatoxin B1, and the scope of application is limited.

Therefore, it is necessary to screen a microbial strain that can efficiently degrade Aflatoxin B1 at a wider range of temperature and pH conditions.

### SUMMARY

The purpose of the present disclosure is to provide an application of Loigolactobacillus coryniformis subsp. coryniformis in efficient degradation of aflatoxin. The fermentation supernatant of Loigolactobacillus coryniformis subsp. coryniformis may effectively degrade Aflatoxin B1, and the highest degradation rate can reach 88.13%. The strain has good acid resistance and can play a better degradation effect under the condition of pH 5.0-7.0. Meanwhile, it has good heat resistance and can still play a role in degradation at 50-55° C., which solves the problem that other microorganisms cannot efficiently play a role in degradation under acidic and high-temperature conditions. The technical scheme adopted by the present disclosure is as follows:

The present disclosure provides an application of Loigolactobacillus coryniformis subsp. coryniformis in an efficient degradation of aflatoxin.

In some embodiments, the Loigolactobacillus coryniformis subsp. coryniformis is Loigolactobacillus coryniformis subsp. coryniformis 523L5, which is preserved in China General Microbiological Culture Collection and Management Center; the preservation number is CGMCC No. 31070, and the preservation date is Jun. 24, 2024; the preservation site is Room 3, No. 1 Yard, Beichen West Road, Chaoyang District, Beijing.

In some embodiments, the application method is as follows: inoculating Loigolactobacillus coryniformis subsp. coryniformis 523L5 into LB broth containing Aflatoxin B1, and degrading Aflatoxin B1 by dark reaction at 25-55° C. and pH 3.0-9.0.

In some embodiments, the concentration of Aflatoxin B1 in LB broth is 0.05-2 μg/mL.

In some embodiments, the dark reaction conditions are 37° C. and pH 7.0 for 48 h.

In some embodiments, the Loigolactobacillus coryniformis subsp. coryniformis 523L5 is inoculated in LB broth before inoculation, cultured in a shaker at 37° C. for 24 h for activation, and inoculated into LB broth containing Aflatoxin B1 at a volume concentration of 1%.

Compared with the existing technology, the beneficial effect of the present disclosure is mainly reflected in:

The Loigolactobacillus coryniformis subsp. coryniformis 523L5 has good growth performance, high acid production ability, strong salt tolerance, and acid tolerance, and it may tolerate about 7% salinity. It may grow at pH 4.5 and has good fermentation performance. The fermentation broth can efficiently degrade Aflatoxin B1 under acidic (pH 5.0-7.0) and high temperature (50-55° C.) conditions, and the highest degradation rate can reach 88.13% at 40° C. and pH 7.0.

### BRIEF DESCRIPTION OF THE DRAWINGS

FIG. 1. Colony (a) and cell morphology (b) of strain 523L5.

FIG. 2. Phylogenetic tree of strain 523L5.

FIG. 3. Growth curve of strain 523L5 in LB medium and LB medium containing Aflatoxin B1.

FIG. 4. Growth curve of strain 523L5 under different pH conditions.

FIG. 5. Single colony of strain 523L5 on solid medium with coumarin as the sole carbon source.

FIG. 6. High-performance HPLC of strain 523L5 before and after the removal of Aflatoxin B1; the peak of Aflatoxin B1 is at 20 min.

FIG. 7. Degradation rate of strain 523L5 on Aflatoxin B1 under different pH conditions.

FIG. 8. Degradation rate of Aflatoxin B1 by strain 523L5 under different temperature conditions.

FIG. 9. Degradation rate of Aflatoxin B1 by strain 523L5 over time.

FIG. 10. Degradation rate of strain 523L5 on Aflatoxin B1 with different concentrations.

FIG. 11. Degradation rate of Aflatoxin B1 by the fermentation supernatant of strain 523L5 and its heat treatment and protease K treatment.

### DETAILED DESCRIPTION OF THE EMBODIMENTS

The following is a better description of the present disclosure in combination with concrete implementation examples, but the protection range of the present disclosure is not limited to this:

The experimental methods in the following implementation examples, if not specifically described, are all conventional methods; the materials and reagents used are obtained from commercial sources without special instructions.

The medium used is as follows:
1) MRS agar: 10.0 g of peptone, 8.0 g of beef extract powder, 5.0 g of yeast extract powder, 20.0 g of glucose, 2.0 g of dipotassium hydrogen phosphate, 2.0 g of diammonium hydrogen citrate, 5.0 g of sodium acetate, 0.2 g of magnesium sulfate, 0.04 g of manganese sulfate, 1.0 mL of Tween80, 14.0 g of agar, pH 6.5±0.2, dissolved in 1000 mL of distilled water, sterilized at 121° C. for 15 min.
2) Coumarin solid medium: 0.25 g of coumarin, 0.25 g of ammonium sulfate, 0.625 g of potassium dihydrogen phosphate, 0.0625 g of magnesium sulfate heptahydrate, 0.125 g of disodium hydrogen phosphate, 0.025 g of calcium chloride, 0.25 mg of ferrous sulfate heptahydrate, 5 g of agar, dissolved in 250 mL of distilled water, high-pressure sterilized at 121° C. for 15 min, poured to a plate for cooling.
3) LB broth: 10.0 g of tryptone, 5.0 g of yeast extract, 10.0 g of sodium chloride, dissolved in 1000 mL of distilled water, high-pressure sterilized at 121° C. for 15 min.
4) Nitrite degradation test medium: 2 mL of 5 mg/mL NaNO₂ aqueous solution is added to 50 mL of MRS medium.

### Example 1: Screening and Identification of Loigolactobacillus coryniformis subsp. coryniformis 523L5

### 1. Preparation of “Qianlipiaoxiang” Pickles

Fresh mustard stems are washed in flowing tap water, drained, cut into appropriate sizes, boiled, and washed. After cooling, 1.2 kg of mustard stems are weighed and placed in a 3 L pickle jar, and 1% pepper and 1% garlic are added meanwhile. 3% salt solution is added to immerse the mustard stems, and then the jar is sealed and placed at room temperature (25-30° C.) for fermentation for 3 months. After fermentation, the color, texture, aroma, and taste of the fermented pickles are scored by trained team members according to Table 1. The pickles with a total score greater than 70 are defined as excellent fermented pickles and used as samples for the next isolation of strains.

TABLE 1
Scoring criteria

| Index | Scoring criterion | Score |
|---|---|---|
| Color | The fermentation broth is turbid, with poor color and a | 21-25 |
| | floating film on the surface | |
| | The fermentation broth is slightly turbid, and the color is | 11-20 |
| | dark | |
| | The fermentation broth is clear and bright in color | 0-10 |
| Texture | The texture is soft and rotten, and the brittleness is | 21-25 |
| | basically absent | |
| | Soft texture, poor brittleness | 11-20 |
| | Harder texture, better brittleness | 0-10 |
| Aroma | No obvious sour smell, smelly, special flavor rich | 21-25 |
| | Stinky with acid, a little special flavor of the pickles | 11-20 |
| | Not stinky, only sour, or have a bad smell, no special | 0-10 |
| | flavor of the pickles | |
| Taste | It has the proper taste of the pickle, and the taste is | 21-25 |
| | better. | |
| | It has the proper taste of the pickle, but it is acidic or has | 11-20 |
| | an acidic taste, and the taste is poor | |
| | It does not have the taste that the pickles should have, | 0-10 |
| | only sour, or it has a bad smell | |

### 2. Preliminary Screening:

The water of “Qianlipiaoxiang” pickles in Step 1 is diluted with sterile saline in a series of gradient dilutions of 10 times, and 100 μL of 100 (i.e., stock solution), 10-3, and 10-4 dilution samples are collected. The samples are evenly coated on MRS agar plates with sterile coating rods. After 48 h of culture at 37° C., colonies with calcium-dissolving circles and different shapes are selected and repeatedly crossed on MRS agar plates. Finally, single colonies are obtained, recorded as strains 523L1, 523L5, and 523L12. The purified strains are frozen in 20% glycerol.

### 3. Rescreening:

A sterile inoculation ring is used to pick the bacterial solution frozen in the glycerol tube in Step 2, and it is streaked on the MRS agar plate. After incubation at 37° C. for 48 h, a ring of single colonies is picked and mixed in a test tube containing MRS medium. After incubation at 37° C. for 24 h, the bacterial density (OD_{600 nm}) and pH value of the samples are measured at 12 h and 24 h, respectively. The results are shown in Table 2.

2 mL of activated culture medium (OD_{600 nm}=1) is added into 50 mL of sterilized nitrite degradation test medium. After culturing at 37° C. for 70 h, the nitrite content is determined by the hydrochloric acid naphthalene ethylenediamine method, and the nitrite degradation rate of each strain is obtained. The results are shown in Table 2.

Meanwhile, the activated strain is coated in MRS agar containing different mass concentrations (3%, 4%, 5%) of NaCl and a mass concentration of 2% calcium carbonate, respectively, and cultured at 37° C. for 48 h. The calcium-dissolving ring around the colony indicates that the strain has salt tolerance. The results of NaCl concentration tolerance are shown in Table 2.

TABLE 2
Determination results of fermentation performance of each strain

| | OD_{600 nm} | | pH | | Nitrite degradation | Salt |
|---|---|---|---|---|---|---|
| Strain | 12 h | 24 h | 12 h | 24 h | capacity (%) | tolerance |
| 523L1 | 1.35 | 1.96 | 4.57 | 3.83 | 40.58 | <3% |
| 523L5 | 1.67 | 2.06 | 4.33 | 3.91 | 43.36 | >5% |
| 523L12 | 1.63 | 2.03 | 4.14 | 3.75 | 41.96 | 3% |

Among them, strain 523L5 has good growth ability, strong acid production ability, salt tolerance ability, and strong nitrite degradation ability.

### 4. Identification of Strain 523L5

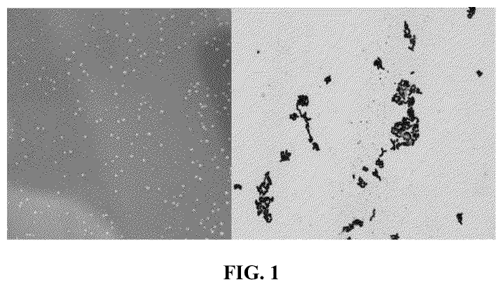

Morphological identification: 100 μL of bacterial solution frozen in a glycerol tube is drawn and streaked on MRS agar plate. After 48 h of culture at 37° C., the colony morphology is observed with the naked eye. After Gram staining, the cell morphology is observed under an optical microscope. The colony and cell morphology of strain 523L5 are shown in FIG. 1. The colony is round, translucent, white, moist, and smooth, and the edge is neat. Under the optical microscope, the strain is blue-purple, gram-positive, rod-shaped.

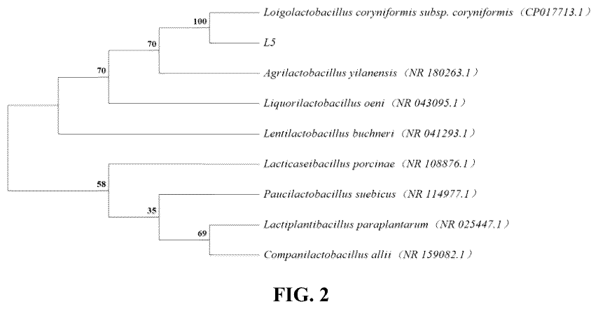

Molecular identification: The screened strains are inoculated in a test tube containing MRS broth, and cultured at 37° C. for 16 h, and the bacteria are harvested. DNA is extracted according to the bacterial genomic DNA extraction kit. The DNA is used as a template, and the bacterial universal primer (27F: 5′-AGAGTTTGATCCTGGCTCAG-3′; 1492R: 5′-TACGGCTACCTTGTTACGACTT-3′). The PCR products are sent to Beijing Qingke Biotechnology Co., Ltd. for sequencing. Basic Local Alignment Search Tool (BLAST) is used for homology comparison, and MEGA10.0 is used to construct a phylogenetic tree, as shown in FIG. 2.

The 16S rDNA sequence (SEQ ID NO.1) is introduced into NCBI for BLAST homology comparison. The results show that the similarity between strain 523L5 and Loigolactobacillus coryniformis subsp. coryniformis is 100%. Combined with morphological characteristics, strain 523L5 is identified as Loigolactobacillus coryniformis subsp. coryniformis, which is preserved in China General Microbiological Culture Collection and Management Center. The preservation site is Room 3, No. 1 Yard, Beichen West Road, Chaoyang District, Beijing; the preservation number is CGMCC No. 31070, and the preservation date is Jun. 24, 2024.

### Example 2: Fermentation Performance Test of Loigolactobacillus coryniformis Subsp. Coryniformis 523L5

### 1. Determination of Growth Curve

The growth ability is an important parameter to evaluate the fermentation agent, and the growth curve can intuitively understand the growth law of the bacteria.

Loigolactobacillus coryniformis subsp. coryniformis 523L5 is inoculated in LB broth and cultured at 37° C. for 24 h. The activated bacteria solution is inoculated into LB broth containing 1 μg/mL Aflatoxin B1 at an inoculum size of 1% (V/V) and cultured at 37° C. for 72 h. Samples are taken every 1 h during 0-7 h and 25-48 h, and samples are taken every 15 min during the intermediate logarithmic growth phase (7-25 h) to determine the bacterial density of the samples (OD_{600 nm}). Under the same conditions, the LB broth containing 1 μg/mL Aflatoxin B1 is changed to LB broth, and other operations are the same.

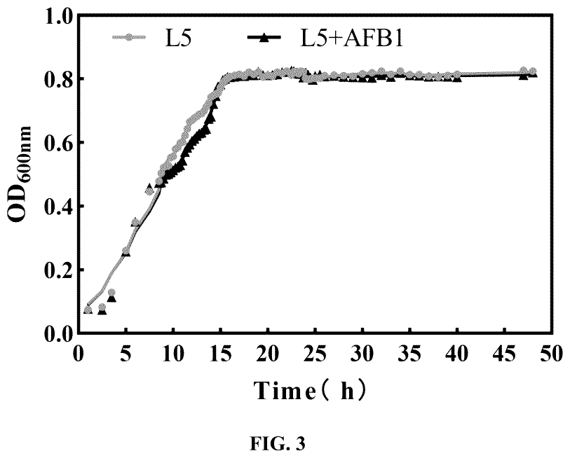

As shown in FIG. 3, at 0-4 h, Loigolactobacillus coryniformis subsp. coryniformis 523L5 is in the growth lag phase and grows slowly. After about 6 h, it enters the logarithmic growth period, and the number of bacteria increases rapidly. After about 15 h, the growth is slow and begins to enter the stable period. The growth status in the medium containing Aflatoxin B1 is similar to that without Aflatoxin B1.

Therefore, Loigolactobacillus coryniformis subsp. coryniformis 523L5 has good growth ability, and the presence of Aflatoxin B1 does not affect its growth.

### 2. The Effect of pH on the Growth of Strain 523L5

The strain 523L5 is activated in LB broth at 37° C. for 24 h, and then inoculated into LB broth at pH 3.0, 4.0, 5.0, 6.0, 7.0, 8.0, and 9.0 with 1% (V/V) inoculation amount. After 72 h of static culture at 37° C., samples are taken every 1 h in 0-7 h and 25-48 h, and samples are taken every 15 min in the middle logarithmic growth phase (7-25 h) to determine the bacterial density (OD_{600 nm}) of the samples.

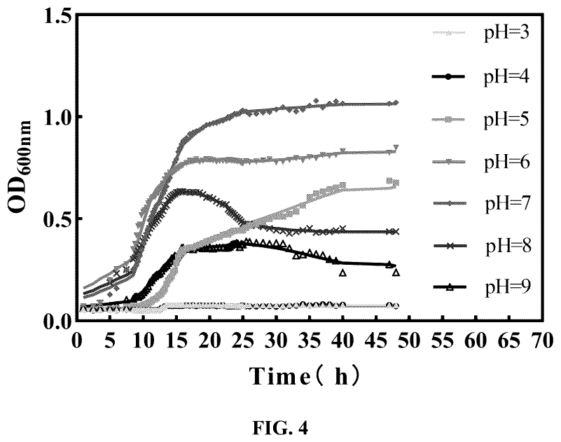

As shown in FIG. 4, when the pH is less than 4.0, Lactobacillus clavatus 523L5 may hardly grow normally. When the pH increases to 5.0, the inhibition of Loigolactobacillus coryniformis subsp. coryniformis 523L5 is less. When the pH rises to more than 6.0, Loigolactobacillus coryniformis subsp. coryniformis 523L5 may grow normally, but when the pH reaches more than 8.0, that is, under alkaline conditions, its growth is significantly lower than that of pH 6.0 and 7.0.

This indicates that the strain 523L5 has better acid resistance, and the growth condition in acidic neutral conditions is better than that in alkaline conditions.

Example 3, the application of Loigolactobacillus coryniformis subsp. coryniformis 523L5 in the degradation of Aflatoxin B1

### 1. The Growth Ability of Strain 523L5s on Coumarin Medium

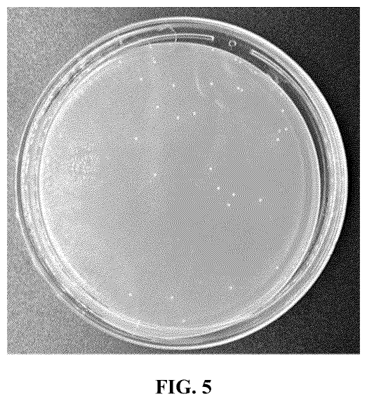

The single colony of Loigolactobacillus coryniformis subsp. coryniformis 523L5 screened and identified in Example 1 is inoculated into LB broth and cultured in a shaker at 37° C. for 48 h. After full enrichment, 200 μL of the bacterial solution obtained from the enrichment culture is coated on a coumarin agar with a coumarin concentration of 1 g/L, and cultured at 37° C. for 3-7 days until visible colonies appear. The single colonies are isolated and transferred to a fresh coumarin agar plate for repeated culture 3 times to obtain a single colony that may grow with coumarin as a carbon source, as shown in FIG. 5.

### 2. Identification of the Degradation Ability of Aflatoxin B1 by Strain 523L5

Lactobacillus coryniformis subsp. coryniformis 523L5 is inoculated in LB broth, activated by shaking at 37° C. for 24 h, and inoculated into LB medium containing 1 μg/mL Aflatoxin B1 and pH 7.0 according to 1% (V/V) inoculation amount. The non-inoculated bacteria are used as the control group, and the reaction is carried out in the shaker at 37° C. for 36 h, with a total of five groups in parallel. After the reaction, the supernatant is collected by centrifugation, and the supernatant is vortexed with an equal volume of methanol for 60 s. After filtration with a 0.22 μm filter membrane, the filtrate is detected by high-performance liquid chromatography (HPLC) for Aflatoxin B1 content.

HPLC conditions are as follows: mobile phase A is pure water, mobile phase B is a mixture of methanol and acetonitrile at a volume ratio of 1:1, and the volume ratio of phase A to phase B is 50:50; the chromatographic column is C18 column; the flow rate is 0.8 mL/min. Injection volume: 50 μL; column temperature: 40° C., excitation wavelength: 360 nm, emission wavelength: 440 nm.

$$Aflatoxin B 1 degradation rate = \frac{{A}_{0} - {A}_{1}}{{A}_{0}} \times 100 \%$$

Where A₀ is the content of Aflatoxin B1 in the control group; and A₁ is the content of Aflatoxin B1 in the experimental group.

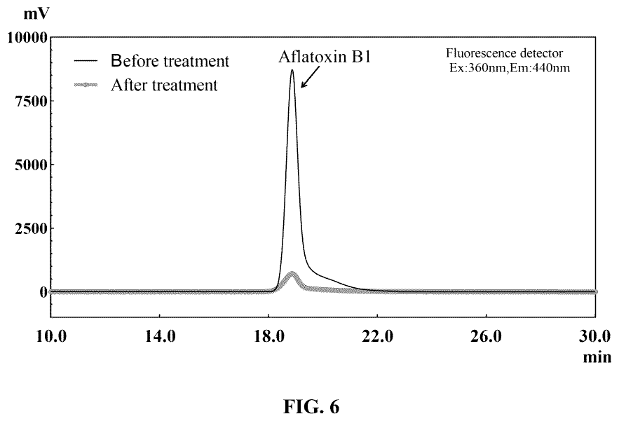

FIG. 6 shows the HPLC of strain 523L5 before and after degradation of Aflatoxin B1, and the peak time of Aflatoxin B1 is about 20 min. After 24 h, the degradation rate of Aflatoxin B1 reaches 52.20%.

### 3. The Effects of Different pH Conditions on the Degradation of Aflatoxin B1 by Strain 523L5

The strain 523L5 is inoculated in LB broth, activated by shaking at 37° C. for 24 h, and inoculated with 1% (v/v) inoculation amount to LB medium containing 1 μg/mL Aflatoxin B1 and pH 3.0, 4.0, 5.0, 6.0, 7.0, 8.0, and 9.0, respectively. The non-inoculated bacteria are used as the control group, and the reaction is carried out in the shaker at 37° C. for 24 h. After the reaction, the supernatant is collected by centrifugation and vortexed with an equal volume of methanol for 60 s. After filtration with a 0.22 μm filter membrane, the filtrate is used to detect the content and degradation rate of Aflatoxin B1 by liquid chromatography.

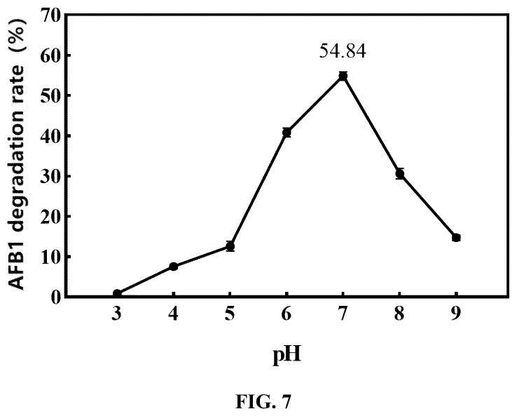

As shown in FIG. 7, under the condition of pH less than 4.0, there is almost no degradation effect on Aflatoxin B1. After pH>5.0, with the increase of pH value, the degradation rate of Aflatoxin B1 shows an upward trend, reaching a maximum of 54.84% at pH 7.0. When the pH is greater than 8.0, the degradation effect of Aflatoxin B1 shows a significant downward trend.

### 4. The Effects of Different Temperature Conditions on the Degradation of Aflatoxin B1 by Strain 523L5

The strain 523L5 is inoculated in LB broth, activated by shaking at 37° C. for 24 h, and inoculated into LB medium containing 1 μg/mL Aflatoxin B1 and pH 7.0 with 1% (v/v) inoculation amount. The non-inoculated bacteria are used as the control group, and the reaction is carried out in the shaker at 25° C., 30° C., 35° C., 40° C., 45° C., 50° C., and 55° C. for 48 h. After the reaction, the supernatant is collected by centrifugation and vortexed with an equal volume of methanol for 60 s. After filtration with a 0.22 μm filter membrane, the filtrate is used to detect the content of Aflatoxin B1 by HPLC, and the degradation rate is calculated.

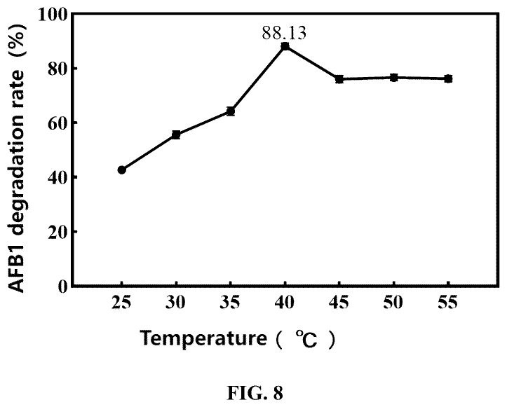

As shown in FIG. 8, when the temperature is lower than 40° C., the degradation rate of Aflatoxin B1 shows an upward trend, reaching a maximum of 88.13% at 40° C. When the temperature is higher than 50° C., the growth of strain 523L5 is inhibited, but it may still have a 76.07% degradation effect, which indicates that the main degradation effect is a heat-resistant active substance in the fermentation broth.

### 5. The Effect of Different Incubation Times on the Degradation of Aflatoxin B1 by Strain 523L5

The strain 523L5 is inoculated in LB broth and cultured at 37° C. for 24 h for activation. The inoculation amount is 1% (v/v) and inoculated into LB medium containing 1 μg/mL Aflatoxin B1 and pH 7.0. The uninoculated bacteria are used as the control group, and the reaction is carried out in the shaker at 37° C. for 12 h, 24 h, 36 h, 48 h, 60 h, and 72 h, respectively. After the reaction, the supernatant is collected by centrifugation and vortexed with an equal volume of methanol for 60 s. After filtration with a 0.22 μm filter membrane, the filtrate is used to detect the content of Aflatoxin B1 by HPLC, and the degradation rate is calculated.

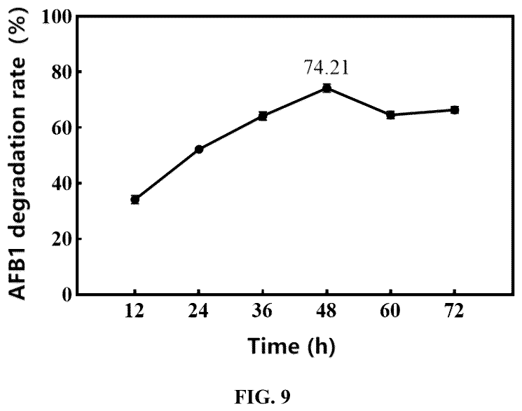

As shown in FIG. 9, with the increase of incubation time, the degradation rate of Aflatoxin B1 by strain 523L5 shows an upward trend, reaching a maximum of 74.21% at 48 h. After 60 h, the degradation rate of Aflatoxin B1 shows a slight downward trend, which may be that the growth of strain 523L5 is inhibited, and part of the degradation effect comes from bacterial adsorption.

The reaction solution of 24 h, 48 h, 72 h is sampled and centrifuged, and the precipitate is resuspended with methanol. The precipitate is ultrasonically broken at 300 W for 10 min. After centrifugation again, the supernatant is taken to detect the content of Aflatoxin B1. The results are shown in Table 3.

TABLE 3
The content of Aflatoxin B1 in 523L5 cell
precipitate after 24 h, 48 h, and 72 h.

| Treatment time | Aflatoxin B1 content (μg/mL) |
|---|---|
| 24 h | 0.1324 |
| 48 h | 0.2046 |
| 72 h | 0.0598 |

As shown in Table 3, 523L5 bacteria have a small amount of bacterial adsorption, and it is significantly reduced at 72 h.

### 6. The Degradation Effect of Strain 523L5 on Different Concentrations of Aflatoxin B1

The strain 523L5 is inoculated in LB broth and activated by shaking at 37° C. for 24 h. According to the inoculation amount of 1% (v/v), it is inoculated into LB medium containing 0.05 μg/mL, 0.1 μg/mL, 0.5 μg/mL, 1 μg/mL, 1.5 μg/mL and 2 μg/mL Aflatoxin B1 and pH 7.0. The non-inoculated bacteria are used as the control group, and the reaction is carried out in the shaker at 37° C. for 24 h. After the reaction, the supernatant is collected by centrifugation, and the supernatant is mixed with an equal volume of methanol vortex for 60 s. After filtration with a 0.22 μm filter membrane, the filtrate is detected by HPLC to detect the content of Aflatoxin B1, and the degradation rate is calculated.

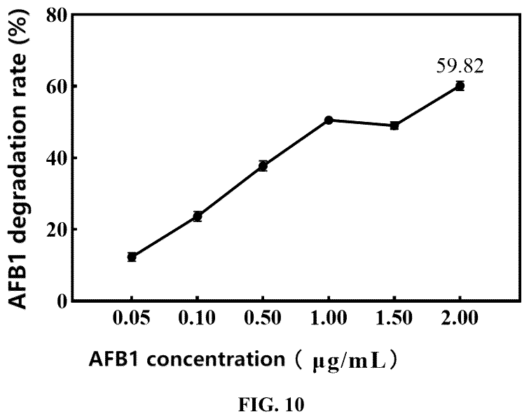

As shown in FIG. 10, with the increase of Aflatoxin B1 concentration, the ability of strain 523L5 in degrading Aflatoxin B1 shows an upward trend, and the degradation rate of 2 μg/mL Aflatoxin B1 reaches a maximum of 59.82%.

### 7. The Fermentation Supernatant of Strain 523L5 and its Degradation Effect on Aflatoxin B1 Under the Treatment of Protease K

The strain 523L5 is inoculated in LB broth, activated by shaking at 37° C. for 24 h, and inoculated into LB medium containing 1 μg/mL Aflatoxin B1 and pH 7.0 with 1% (v/v) inoculation amount. The reaction is carried out in a shaker at 37° C. for 48 h in the dark. After centrifugation at 10000 rpm for 10 min, the fermentation supernatant and bacterial precipitate are collected, respectively. The precipitate is resuspended with 5 times the volume of 1 M PBS buffer. The 2 mL of fermentation supernatant is taken out and kept in a 5 mL centrifuge tube for 20 min in a boiling water bath to obtain the thermally denatured supernatant. Then, 2 mL of fermentation supernatant is taken into a 5 mL centrifuge tube, and 1 μg/mL proteinase K is added. The supernatant is mixed and placed at room temperature for 20 min to obtain the supernatant treated with proteinase K.

1 mL of PBS buffer is added with 1 μg/mL Aflatoxin B1, and then 50 μL of fermentation supernatant (denoted as FS), thermal denaturation supernatant (denoted as FS-T), protease K-treated supernatant (denoted as FS-PrK), and resuspended cell precipitate (denoted as cell) are added, respectively. After fully mixing, the reaction is carried out in a shaker at 37° C. for 24 h in the dark. After the reaction, the supernatant is collected by centrifugation and vortexed with an equal volume of methanol for 60 s. After filtration with a 0.22 μm filter membrane, the filtrate is used to detect the content of Aflatoxin B1 by HPLC, and the degradation rate is calculated.

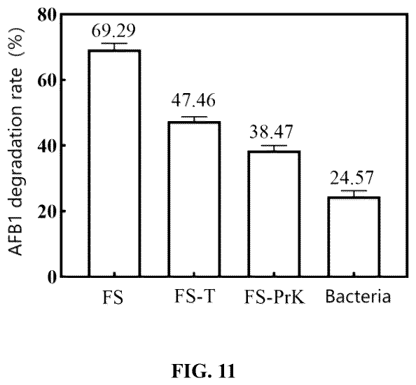

As shown in FIG. 11, the main active ingredient of strain 523L5 degrading Aflatoxin B1 is in the fermentation supernatant, and the degradation rate is as high as 69.29%. After thermal denaturation treatment and protease K treatment, the degradation rate decreases significantly, indicating that some protein substances or enzymes played a role in degradation in the fermentation supernatant.

What is claimed is:

1. A method of use of Loigolactobacillus coryniformis subsp. coryniformis in an efficient degradation of aflatoxin, wherein the Loigolactobacillus coryniformis subsp. coryniformis is Loigolactobacillus coryniformis subsp. coryniformis 523L5, and wherein the preservation number is CGMCC No. 31070; and wherein the method comprises a step of culturing the Loigolactobacillus coryniformis subsp. coryniformis.

2. The method according to claim 1, wherein the method comprises a step of: inoculating Loigolactobacillus coryniformis subsp. coryniformis 523L5 into LB broth containing Aflatoxin B1, and degrading Aflatoxin B1 by dark reaction at 25-55° C. and pH 3.0-9.0.

3. The method according to claim 2, wherein the concentration of Aflatoxin B1 in LB broth is 0.05-2 μg/mL.

4. The method according to claim 2, wherein the dark reaction conditions are 37° C. and pH 7.0 for 48 h.

5. The method according to claim 2, wherein the Loigolactobacillus coryniformis subsp. coryniformis 523L5 is inoculated in LB broth before inoculation, cultured in a shaker at 37° C. for 24 h for activation, and inoculated into LB broth containing Aflatoxin B1 at a volume concentration of 1%.
